(19) United States
(12) Patent Application Publication
(10) Pub. No.: US 2004/0013617 A1
(43) Pub. Date: Jan. 22, 2004

(54) Sunless tanning composition

(76) Inventors: Donald Rick, Dumont, NJ (US); Isabelle Hansenne, Westfield, NJ (US); A. John Penicnak, Boonton Township, NJ (US)

Correspondence Address: Thomas L. Irving, FINNEGAN HENDERSON, FARABOW,, GARRETT & DUNNER, L.L.P., 1300 I Street, N.W., Washington, DC, 20005-3315, US

(21) Appl. No.: 10/198,116

(22) Filed: Jul. 19, 2002

(51) Int. Cl. A61K 7/42

(52) U.S. Cl. 424/59

(57) ABSTRACT

The present invention relates to a sunless tanning composition comprising at least one sunless tanning active ingredient, at least one phospholipid, at least one nonionic surfactant, and at least one amphoteric surfactant. The sunless tanning active ingredient cab be dihydroxyacetone or erythrulose. The composition can, for example, be provided as clear, aqueous-based formulations that can be incorporated into sunless tanning sprays and non-aerosol mousses. Applying the composition to skin may provide enhanced tanning, such as enhanced tanning compared to a formula having the same level of sunless tanning active ingredient but without the phospholipid, nonionic surfactant, and amphoteric surfactant.

The present invention relates to sunless tanning compositions comprising at least one sunless tanning active ingredient, at least one phospholipid, at least one nonionic surfactant, and at least one amphoteric surfactant. The present invention also relates to methods of making such compositions and to methods of sunless tanning.

A tanned appearance is aesthetically pleasing to certain individuals. It is well established, however, that excessive exposure to sunlight is detrimental to the skin and can result in skin damage ranging from mild sunburn to skin cancer. Thus, sunless tanning compositions have been developed as a safer alternative to obtaining a natural tan.

Dihydroxyacetone (DHA) has been widely used as an active ingredient in sunless tanning compositions. It has been reported that DHA reacts with certain nitrogen-containing compounds in the skin, imparting the skin with a tanned color. DHA, however, provides many disadvantages, particularly its instability in solution, resulting in a short shelf life, and lengthy times to achieve the desired tanned color. Attempts to overcome such disadvantages include improving the chemical stability of DHA, or adding waterproofing polymers. Such attempts, however, did not result in an improved tanned color on the skin, and/or did not lengthen the time of the resulting tanned color on the skin.

The inventors have surprisingly discovered that a composition comprising at least one phospholipid, at least one nonionic surfactant, at least one amphoteric surfactant, and at least one sunless tanning active ingredient provides an enhanced tanned result.

In one embodiment of the present invention, the at least one sunless tanning active ingredient is dihydroxyacetone. In another embodiment of the present invention, the at least one sunless tanning active ingredient is dihydroxyacetone and the at least one phospholipid is lecithin.

Another aspect of the present invention relates to a method of making a sunless tanning composition comprising combining at least one phospholipid, at least one nonionic surfactant, at least one amphoteric surfactant, and at least one sunless tanning active ingredient.

In another aspect, the present invention provides a method of sunless tanning comprising applying to skin a composition comprising at least one phospholipid, at least one nonionic surfactant, at least one amphoteric surfactant, and at least one sunless tanning active ingredient.

Yet another aspect of the present invention provides a composition comprising at least one phospholipid, at least one nonionic surfactant, at least one amphoteric surfactant, and at least one dihydroxyacetone, wherein the composition is an aqueous-based solution.

In one embodiment, the compositions of the invention provide enhanced tanning. For example, the composition can be capable of darkening skin by a ΔE value of at least 0.05, compared to a composition free of a mixture comprising at least one phospholipid, at least one nonionic surfactant, and at least one amphoteric surfactant, within a time period ranging from 5 hours to 72 hours upon applying the composition to skin. In another example, the composition can be capable of darkening skin by a Δa value of at least 0.05, compared to a composition free of a mixture comprising at least one phospholipid, at least one nonionic surfactant, and at least one amphoteric surfactant, within a time period ranging from 5 hours to 72 hours upon applying the composition to skin.

It is to be understood that both the foregoing general description and the following detailed description are exemplary and explanatory only and are not restrictive of the invention, as claimed.

### BRIEF DESCRIPTION OF THE DRAWINGS

FIG. 1 is a graph of average ΔE (y-axis) vs. time (x-axis) for a DHA-containing formulation without a LAN delivery vehicle (Formula 1) and with a LAN delivery vehicle (Formula 2); and

FIG. 2 is a graph of average Δa (y-axis) vs. time (x-axis) for a DHA-containing formulation without a LAN delivery vehicle (Formula 1) and with a LAN delivery vehicle (Formula 2).

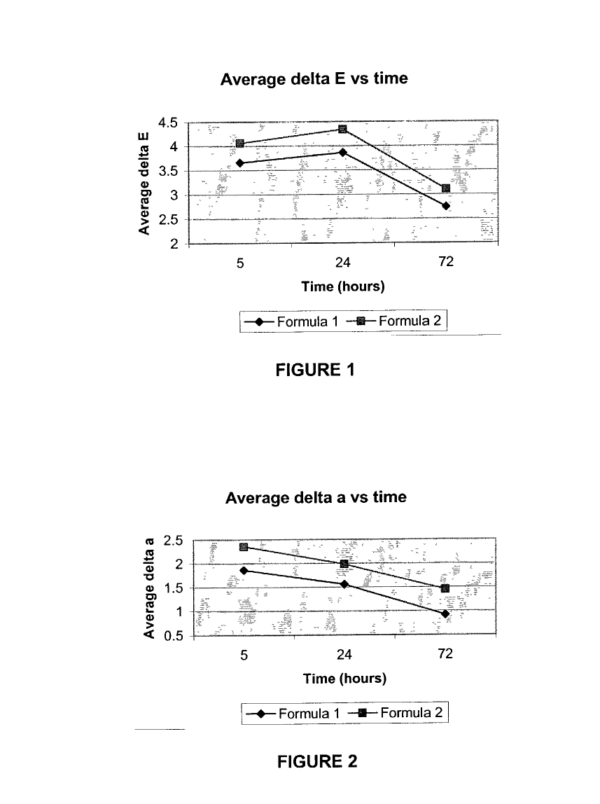

In one embodiment, the present invention provides sunless tanning compositions comprising at least one sunless tanning active ingredient, at least one phospholipid, at least one nonionic surfactant, and at least one amphoteric surfactant.

The combination of phospholipid, nonionic surfactant, and amphoteric surfactant has been previously reported in U.S. Pat. Nos. 6,015,574 and 6,221,389, as a delivery system for lipophilic (oil-soluble) materials in an aqueous solution. The present inventors have discovered that compositions comprising a sunless tanning active ingredient, such as DHA or erythrulose, which are water-soluble, result in a formulation that can offer enhanced tanning results when combined with at least one phospholipid, at least one nonionic surfactant, and at least one amphoteric surfactant. The combination of at least one phospholipid, at least one nonionic surfactant, and at least one amphoteric surfactant is referred to herein as a “delivery vehicle.”

Thus, in one embodiment, the present compositions provide a delivery vehicle for enhanced delivery of the sunless tanning active ingredient to the skin. This enhanced delivery can produce a darker and longer lasting tan.

As used herein, an “enhanced tanning result” refers to at least one of the following improvements over a composition including a sunless tanning active ingredient, such as DHA or erythrulose, but without the present delivery vehicle as measured using the procedure of the Example: (1) a darker tan, as indicated by an increased ΔE value; (2) a redder and thus, a more natural looking tan, as indicated by an increased Δa value; and (3) a longer lasting tan. ΔE is a measure of the total change in color of skin before and after the present composition is applied, with a larger ΔE value indicating more color has developed resulting in a darker tan. ΔE is mathematically described as:

ΔE=[(ΔL)²+(Δa)²+(Δb)²]^{1/2}

This formula applies where measurements are taken that describe the color in L*a*b* color space. In this color space, L* indicates lightness and a* and b* are the chromaticity coordinates. The a* and b* indicate color directions: +a* is the red direction, −a* is the green direction, +b* is the yellow direction, and −b* is the blue direction.

In some cases, the application of a sunless tanning active ingredient, such as DHA or erythrulose, can result in an unnatural color on the skin, such as an orange-like color. Thus, a redder color tan may be desired to provide a more natural looking tan. Δa is a measure of the red color development, where a larger Δa value indicates that a stronger red color has developed from the tan.

A “sunless tanning active ingredient” is one that, when applied to skin, is capable of imparting a darker tan, as defined above, to skin via a chemical reaction with components in the skin. In one embodiment, the at least one sunless tanning active ingredient is chosen from DHA and erythrulose.

In one embodiment, the at least one phospholipid is a lecithin. Lecithins are mixtures of phospholipids, i.e., of diglycerides of fatty acids linked to an ester of phosphoric acid. Lecithins can be, for example, diglycerides of stearic, palmitic, and oleic acids linked to the choline ester of phosphoric acid. In one embodiment, lecithins can be either pure phosphatidyl cholines or crude mixtures of phospholipids, which include phosphatidyl choline, phosphatidyl serine, phosphatidyl ethanolamine, phosphatidyl inositol, other phospholipids, and a variety of other compounds such as fatty acids, triglycerides, sterols, carbohydrates, and glycolipids.

The lecithin used in the present invention, for example, may be present in the form of a liquid, powder, or granules. Lecithins useful in certain embodiments of the invention include, but are not limited to, soy lecithin and hydroxylated lecithin. For example, ALCOLEC S is a fluid soy lecithin, ALCOLEC F100 is a powder soy lecithin, and ALCOLEC Z3 is a hydroxylated lecithin, all of which are available from the American Lecithin Company.

In one embodiment where the phospholipid is a lecithin, the delivery vehicle is referred to herein as “LAN,” i.e., lecithin (L) as the phospholipid, an amphoteric surfactant (A), and a nonionic surfactant (N). In one embodiment, LAN compositions can resist storage at 45° C. for three months or more, which would predict that they have a shelf life at room temperature of at least three years.

The amphoteric surfactants useful in the present invention include, but are not limited to, betaines, sultaines, hydroxysultaines, alkyl amphodiacetates, alkyl amphodipropionates, and imidazolines, or salts thereof. It is recognized that other fatty acid condensates such as those formed with amino acids, proteins, and the like are also suitable. Amphoteric surfactants are typically available for commercial sale in solution form with the active surfactant accounting for approximately 40% of the total solution weight. Cocamphodipropionate can also be used, such as, for example, MIRANOL C2M-SF Conc. (disodium cocamphodipropionate), in its salt-free form, available from Rhône-Poulenc. Alternatively, disodium cocamphodipropionate, supplied by McIntyre Group Ltd. under the trade name MACKAM 2 CSF 40GG can be used. MIRANOL is sold in solution form with amphoteric surfactants composing approximately 40% of the total solution weight; for example, 10 g of MIRANOL contain about 4 g of amphoteric surfactant. Another exemplary amphoteric surfactant is CROSULTAINE C-50 (cocamidopropyl hydroxysultaine), available from Croda. CROSULTAINE is also sold in solution form with the amphoteric surfactant composing approximately 50% of the total solution weight. Other amphoteric surfactants useful in the present invention include disodium wheatgermimido PEG-2 sulfosuccinate, available under the trade name MACKANATE WGD from McIntyre Group Ltd., which is a solution with amphoteric surfactants composing approximately 39% of the total solution weight, and disodium soyamphodiacetate, available under the trade name MACKAM 2S from McIntyre Group Ltd., which is a solution with amphoteric surfactants composing approximately 34.5% of the total solution weight.

In one embodiment, the nonionic surfactants useful in the present invention can be formed from a fatty alcohol, a fatty acid, or a glyceride with a C₈ to C₂₄ carbon chain, such as a C₁₂ to C₁₈ carbon chain or a C₁₆ to C₁₈ carbon chain, derivatized to yield a Hydrophilic-Lipophilic Balance (HLB) of at least 10. In one embodiment, the nonionic surfactant comprises a residue of a compound chosen from C₈ to C₂₄ fatty alcohols, C₈ to C₂₄ fatty acids, and C₈ to C₂₄ glycerides. “Residue” is referred to herein as the portion of the compound remaining in the desired product. For example, a residue of an alcohol is an alkoxy. HLB is understood to mean the balance between the size and strength of the hydrophilic group and the size and strength of the lipophilic group of the surfactant. Such derivatives can be polymers such as ethoxylates, propoxylates, polyglucosides, polyglycerins, polylactates, polyglycolates, polysorbates, and others that would be apparent to one of ordinary skill in the art. Such derivatives may also be mixed polymers of the above, such as ethoxylate/propoxylate species, where the total HLB can be greater than or equal to 10. The nonionic surfactants can contain ethoxylate in a molar content ranging from 10 to 25, such as a molar content ranging from 10 to 20 moles.

Nonionic surfactants may be selected from, but are not limited to, the following:

| # of C's | Name | Trade Name |
|---|---|---|
| C-12 | Laureth-23 | BRIJ 35, available from ICI Surfactants |
| C-16 | Ceteth-10 | BRIJ 56, available from ICI Surfactants |
| C-16 | Ceteth-20 | BRIJ 58, available from ICI Surfactants |
| C-16 | IsoCeteth-20 | Arlasolve 200, available from ICI Surfactants |
| C-18 | Steareth-10 | Volpo S-10, available from Croda Chemicals Ltd. |
| C-18 | Steareth-16 | Solulan-16, available from Amerchol Corp. |
| C-18 | Steareth-20 | BRIJ 78, available from ICI Surfactants |
| C-18 | Steareth-25 | Solulan-25, available from Amerchol Corp. |
| C-18 | Oleth-10 | BRIJ 97, available from ICI Surfactants |
| C-18 | Oleth-20 | Volpo-20, available from Croda Chemicals Ltd. |

Alkyl polyglucose surfactants sold under the name PLANTAREN, available from Henkel, may also be used.

In one embodiment, the phospholipid, amphoteric surfactant and nonionic surfactant are present in the composition in a ratio of 1:0.8:2 and above by weight, i.e., where the amounts of the surfactants can be increased independently of each other but the amount of phospholipid stays fixed. The ratio is considered to be “above” 1:0.8:2 when the amount of either of the surfactants increases. For example, phospholipid/amphoteric/ nonionic ratio can range from 1:1:2 and above, and from 1:1:4 and above. In one embodiment, the composition of the present invention comprises ALCOLEC F100 (a powdered soy lecithin), MACKAM 2 CSF 40GG (disodium cocamphodipropionate, an amphoteric surfactant), and ARLASOLVE 200 (IsoCeteth-20, a nonionic surfactant) in a ratio of 1:1.2:4 for a DHA-containing solution, where the ratio is calculated by weight. Alternately stated, a LAN ratio of 1:1.2:4 is equal to 5 g lecithin, 6 g MIRANOL, and 20 g ARLASOLVE.

In one embodiment, the present delivery vehicle can be present in an amount equal to or greater than 1% by weight relative to the total weight of the composition. In one embodiment, the delivery vehicle is present in an amount ranging from 1% to 50% by weight relative to the total weight of the composition.

In one embodiment, the sunless tanning active ingredient, such as DHA or erythrulose is present in the composition in an amount ranging from 1% to 50% by weight relative to the total weight of the composition, such as an amount ranging from 1% to 25% by weight or from 2.5% to 8%, or from 3% to 7% by weight, relative to the total weight of the composition.

In one embodiment, the at least one phospholipid, such as lecithin, can be present in an amount ranging from greater than 0% to 25% by weight relative to the weight of the delivery vehicle, such as an amount ranging from 1% to 20% by weight relative to the weight of the delivery vehicle. In another embodiment, the phospholipid, such as lecithin, is present in an amount ranging from 0 to 5% by weight relative to the weight of the composition, such as an amount greater than 0 to 3% by weight relative to the weight of the composition.

The at least one amphoteric surfactant can, for example, be present in the composition in an amount ranging from greater than 0 to 25% by weight relative to the weight of the delivery vehicle, such as an amount greater than 0 to 15% by weight relative to the weight of the delivery vehicle. In one embodiment, the at least one amphoteric surfactant is present in the composition in an amount ranging from 0 to 15% by weight relative to the weight of the composition, such as an amount greater than 1 to 10% by weight relative to the weight of the composition.

The at least one nonionic surfactant can, for example, be present in an amount ranging from greater than 0 to 80% by weight relative to the weight of the delivery vehicle, such as an amount ranging from greater than 0 to 70% by weight relative to the weight of the delivery vehicle. In one embodiment, the at least one nonionic surfactant is present in the composition in an amount ranging from 0 to 25% by weight relative to the weight of the composition, such as an amount greater than 1 to 10% by weight relative to the weight of the composition.

In one embodiment, the present composition can be provided in the form of a nonaerosol mousse, a spray, a gel, a cream, or a lotion.

In one embodiment, the present composition can further comprise at least one anionic surfactant, such as alkyl sulfates and ethoxylated alkyl sulfates. Other anionic surfactants such as sulfosuccinates may also be used.

Other ingredients in the present composition may, for example, include isoparaffins; sodium chloride; propylene glycol; preservatives such as phenoxyethanol, methylparaben, ethylparaben, and propylparaben; pH adjusters such as phosphoric acid; humectants such as trehalose; and emollients such as octyldodecanol. Many other examples of materials from the classes listed above would be readily known to one of ordinary skill in the art.

At least one suspending agent may be present in the composition in an amount ranging from 1% to 20% by weight relative to the total weight of the delivery vehicle. The amount of suspending agent may depend on the viscosity increasing properties of the particular suspending agent. The suspending agents can be present in an amount ranging from 1% to 10% by weight. One of ordinary skill in the can determine routinely the preferred amount of suspending agent depending on the specific delivery vehicle. The suspending agent can be added in an amount effective for maintaining a stable composition or stable delivery vehicle.

Other ingredients that may be present in the composition of the invention include but are not limited to hydrocarbons, waxes, silicones, oil-soluble vitamins such as Vitamin E and Vitamin A, sunscreens, ceramides and natural oils. Exemplary ingredients include: petrolatum, polyethylenes, beeswax, Vitamin E, Vitamin E Acetate, Vitamin A Palmitate, olive oil, mineral oil, 2-oleamido-1,3-octadecanediol, octylmethoxy cinnamate, octyl salicylate, and silicones such as siloxanes, dimethicone, cyclomethicone, phenyl trimethicone, dimethiconol, dimethicone copolyol, and laurylmethicone copolyol. These other ingredients can, for example, moisturize or condition the skin, leaving behind no oily feel.

Other additional ingredients include organic salts, inorganic salts, proteins, water-soluble polymers, quaternary ammonium compounds, complex and simple carbohydrates, amino acids, preservatives and fragrances.

Other ingredients include buffers or other components for regulating pH of the solution, such as citric acid.

In one embodiment, if the present composition is to be used in concentrated form, i.e., with 5% by weight of the phospholipid and greater than 1% of a sunless tanning active ingredient, such as DHA or erythrulose, the composition can have a pH ranging from 2 to 7, or from 4 to 7 for maximum stability. In some embodiments, the more concentrated the solution, the better the delivery. For example, if a blend is diluted with water or the blend is used as an ingredient in another composition, then the pH may have a broader range, such as a range from 2 to 7 or from 4 to 7, and a wider variety of additives may be included in the solution. These dilute blends can still be very effective in delivering sunless tanning active ingredients.

DHA or erythrulose, although water-soluble, are often incorporated in prior art formulations as a water/alcohol formulation or as a water-in-oil or oil-in-water emulsion. In one aspect of the invention, the present delivery vehicle in combination with the sunless tanning active ingredient provides an aqueous-based solution, i.e., where water is the only solvent. In one embodiment, avoiding alcohols or oils as solvent can remove the alcoholic or oily feel that results upon applying the composition to the skin. In another embodiment, the compositions of the invention are water/alcohol formulations or water-in-oil or oil-in-water emulsions. In one embodiment, the solution is clear.

In another embodiment, the composition further comprises a hydroglycolic solution, including solvents such as ethylene glycol and propylene glycol. The hydroglycolic solution can optionally include water.

Another aspect of the present invention provides a method of sunless tanning comprising applying to skin a composition comprising at least one phospholipid, at least one nonionic surfactant; at least one amphoteric surfactant, and at least one sunless tanning composition, such as dihydroxyacetone or erythrulose.

Another aspect of the present invention provides a process for preparing the sunless tanning composition of the present invention. This process comprises: (a) combining the following ingredients to obtain a mixture: at least one phospholipid, at least one amphoteric surfactant, at least one nonionic surfactant, at least one sunless tanning active ingredient, and water, (b) heating and stirring the combined ingredients of (a). Other optional ingredients as described herein can also be added in steps (a) or (b), as can be determined by one of ordinary skill in the art. Either a high shear apparatus or a normal mechanical stirrer may be used for the stirring.

In one embodiment, the mixture can be heated at a temperature of 65° C. to 85° C. in step (b), depending on the melting points of the solid components. In one embodiment, the mixture is heated to about 70° C.

In one embodiment, the preparation of the delivery vehicle of the present invention may be carried out as follows. The phospholipid is dispersed in water. A sunless tanning active ingredient is combined with nonionic surfactant(s) at appropriate ratios and added to the lecithin/water dispersion. An amphoteric surfactant is added and the mixture is heated, while being stirred for about 15 minutes at about 70° C. The combination of these ingredients results in a stable concentrate that can then be used as a “raw material” to make finished products.

Alternatively, the phospholipid(s), amphoteric surfactant(s) and nonionic surfactant(s) can be weighed to appropriate ratios and heated to 70° C. with stirring. Water is then added q.s. at the same temperature. Another alternative method of preparation comprises adding the sunless tanning active ingredient with mixing after solutions have cooled.

Another aspect of the present invention provides a composition comprising at least one phospholipid, at least one nonionic surfactant, at least one amphoteric surfactant; and at least one sunless tanning active ingredient, whereupon after applying the composition to skin, the composition is capable of imparting a darker color and/or a redder color to the skin, compared to a composition containing a sunless tanning active ingredient but without the combination of a phospholipid, nonionic surfactant, and amphoteric surfactant. The darker color can be determined by applying the composition of the invention onto the skin and determining a ΔE value. The ΔE value imparted by the composition of the invention, as measured between five hours and 72 hours after application to the skin, can be greater than a ΔE for a non-inventive composition by at least 0.05. A similar test can be performed to show a higher Δa value imparted by a composition of the invention, where the Δa value afforded by the composition of the invention is at least 0.05 units greater compared to a non-inventive composition, as measured between five hours and 72 hours after application to the skin.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

Unless otherwise indicated, all numbers expressing quantities, reaction conditions, and so forth used herein are to be understood as being modified in all instances by the term “about.” Accordingly, unless indicated to the contrary, the numerical parameters set forth in the specification herein and in the attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

### EXAMPLE

This Example relates to a specific formulation containing DHA and a LAN delivery vehicle provided as sunless tanning non-aerosol mousse. First, a LAN concentrate was made, which was then added to the final formulation.

Lan Concentrate

| Ingredient | % |
|---|---|
| Lecithin | 5.0 |
| Disodium cocoamphodipropionate | 6.0 |
| Isoceteth-20 | 20.0 |
| Water | 69.0 |
| | 100.0 |

Process:

In a beaker, the lecithin, disodium cocoamphodipropionate, and isoceteth-20 were combined and heated to 80° C. until all components were melted together. In a separate beaker, water was heated to 80° C. Water was then added to the LAN ingredients and mixed using an overhead mixer until a homogeneous dispersion was produced. The mixture should be clear when hot, but may become hazy when cool.

Final Non-Aerosol Mousse

| Phase | Ingredient | % |
|---|---|---|
| A1 | Water | to 100% |
| | Preservative | 1.0 |
| | Humectant | 5.0 |
| | Citric acid | 0.15 |
| | Oleth-20 | 2.0 |
| A2 | Water | 15.0 |
| | Dihydroxyacetone | 5.0 |
| B | LAN Concentrate | 10.0 |

Process:

In a beaker, the components of phase A1 were combined and heated to 60° C. The LAN concentrate was added to a separate beaker and mixed with an overhead mixer. Phase A1 was added to the LAN concentrate, producing a clear aqueous solution. The mixture was allowed to cool. In a separate beaker, the components of phase A2 were combined. When the combination of phases Al and B was cooled to room temperature, the solution of phase A2 was added. The complete solution was mixed for 5 minutes.

Tanning Study:

To determine if enhanced tanning occurs in formulations containing the LAN system, the following 2 formulations were compared: Formula 1 (does not contain LAN)

| | |
|---|---|
| Ingredient | % |

Formula 1 (does not contains LAN)

| | |
|---|---|
| Alcohol denatured | 42.5 |
| Humectant | 20.0 |
| Dihydroxyacetone | 5.0 |
| Water | 8.0 |
| Emollient | 23.5 |

Formula 2 (contains LAN)

| | |
|---|---|
| Water | to 100% |
| Preservative | 1.0 |
| Humectant | 20.0 |
| Dihydroxyacetone | 5.0 |
| Lecithin | 0.5 |
| Disodium cocoamphodipropionate | 0.6 |
| Isoceteth-20 | 2.0 |

From Formula 2, it can be seen that the LAN system comprised about 3.1% of the formulation.

Testing Procedure:

18 μL test product was applied in a 3×3 cm square area on each panelist's arm. There were 3 panelists, with each product applied to both arms for a total of 6 test sites for each product. The level of tanning was measured using a Minolta CM-2600d spectrophotometer. For each test site an initial reading was taken before application of test products. After application of the test products, readings were taken at 5, 24, and 72 hours. Readings taken with the Minolta CM-2600d spectrophotometer described the color in L*a*b* color space. In this color space, L* indicates lightness and a* and b* are the chromaticity coordinates. The a* and b* indicate color directions: +a* is the red direction, −a* is the green direction, +b* is the yellow direction, and −b* is the blue direction.

Results:

Average ΔE values for all subjects (3 subjects, 6 sites), are indicated in the table below. A higher value for ΔE indicates more color change, i.e., a darker tan was developed.

| Time (hours) | Formula 1 (non-LAN) | Formula 2 (LAN) |
|---|---|---|
| 5 | 3.66 | 4.06 |
| 24 | 3.86 | 4.34 |
| 72 | 2.74 | 3.10 |

A higher Δa value indicates a stronger red color has developed from the tan, resembling a more natural looking tan. Average Δa values for all subjects (3 subjects, 6 ), are provided in the table below:

| Time (hours) | Formula 1 (non-LAN) | Formula 2 (LAN) |
|---|---|---|
| 5 | 1.86 | 2.35 |
| 24 | 1.56 | 1.98 |
| 72 | 0.92 | 1.45 |

The test product containing the LAN system showed enhanced ΔE and Δa values at each time point as well as a longer lasting tan.

What is claimed is:

1. A composition comprising:
at least one phospholipid;
at least one nonionic surfactant;
at least one amphoteric surfactant; and
at least one sunless tanning active ingredient.

2. A composition according to claim 1, wherein said at least one sunless tanning active ingredient is chosen from at least one dihydroxyacetone and at least one erythrulose.

3. A composition according to claim 1, wherein said composition further comprises water.

4. A composition according to claim 1, wherein said at least one phospholipid is a lecithin.

5. A composition according to claim 4, wherein the lecithin is chosen from soy lecithin and hydroxylated lecithin.

6. A composition according to claim 1, wherein said at least one phospholipid, said at least one amphoteric surfactant, and said at least one nonionic surfactant are present, respectively, in a ratio ranging from 1:1:2, by weight, and above.

7. A composition according to claim 1, wherein said at least one phospholipid, said at least one amphoteric surfactant, and said at least one nonionic surfactant are present in a ratio of 1:1.2:4 by weight.

8. A composition according to claim 1, wherein said at least one nonionic surfactant comprises a residue of a compound chosen from C₈ to C₂₄ fatty alcohols, C₈ to C₂₄ fatty acids, and C₈ to C₂₄ glycerides.

9. A composition according to claim 1, wherein said at least one nonionic surfactant has an HLB of at least 10.

10. A composition according to claim 1, wherein said at least one amphoteric surfactant is chosen from betaines, sultaines, hydroxysultaines, alkyl amphodiacetates, alkyl amphodipropionates, imidazolines, and salts thereof.

11. A composition according to claim 10, wherein said at least one amphoteric surfactant is chosen from cocamphodipropionate and cocamidopropyl hydroxysultaine.

12. A composition according to claim 1, wherein said at least one phospholipid is present in an amount ranging from greater than 0 to 5% by weight relative to the total weight of the composition.

13. A composition according to claim 12, wherein said at least one phospholipid is present in an amount ranging from greater than 0% to 3% by weight relative to the total weight of the composition.

14. A composition according to claim 1, wherein said at least one nonionic surfactant is present in an amount ranging from greater than 0% to 25% by weight relative to the total weight of the composition.

15. A composition according to claim 14, wherein said at least one nonionic surfactant is present in an amount ranging from greater than 0% to 10% by weight relative to the total weight of the composition.

16. A composition according to claim 1, wherein said at least one amphoteric surfactant is present in an amount ranging from greater than 0% to 15% by weight relative to the total weight of the composition.

17. A composition according to claim 16, wherein said at least one amphoteric surfactant is present in an amount ranging from greater than 0% to 15% by weight relative to the total weight of the composition.

18. A composition according to claim 1, wherein the at least one sunless tanning active ingredient is present in an amount ranging from 1% to 50% by weight relative to the total weight of the composition.

19. A composition according to claim 18, wherein at least one sunless tanning active ingredient is present in an amount ranging from 1% to 25% by weight relative to the total weight of the composition.

20. A composition according to claim 19, wherein at least one sunless tanning active ingredient is present in an amount ranging from 2.5% to 8% by weight relative to the total weight of the composition.

21. A composition according to claim 20, wherein at least one sunless tanning active ingredient is present in an amount ranging from 3% to 7% by weight relative to the total weight of the composition.

22. A composition according to claim 1, wherein the pH of said composition ranges from 2 to 7.

23. A composition according to claim 22, wherein said pH ranges from 4 to 7.

24. A composition according to claim 1, wherein said at least one nonionic surfactant is present in an amount by weight equal to or greater than the amount of said at least one phospholipid.

25. A composition according to claim 1, wherein the composition is clear.

26. A composition according to claim 1, wherein the composition is capable of darkening skin by a ΔE value of at least 0.05, compared to a composition free of a mixture comprising at least one phospholipid, at least one nonionic surfactant, and at least one amphoteric surfactant, within a time period ranging from 5 hours to 72 hours upon applying the composition to skin.

27. A composition according to claim 1, wherein the composition is capable of darkening skin by a Δa value of at least 0.05, compared to a composition free of a mixture comprising at least one phospholipid, at least one nonionic surfactant, and at least one amphoteric surfactant, within a time period ranging from 5 hours to 72 hours upon applying the composition to skin.

28. A product chosen from a nonaerosol mousse, a spray, a gel, a cream, and a lotion, comprising the composition according to claim 1.

29. A composition comprising:
at least one lecithin;
at least one nonionic surfactant;
at least one amphoteric surfactant; and
at least one dihydroxyacetone.

30. A composition comprising:
at least one phospholipid;
at least one nonionic surfactant;
at least one amphoteric surfactant; and
at least one dihydroxyacetone.

31. A method of sunless tanning, comprising:
applying to skin, a composition comprising:
at least one phospholipid;
at least one nonionic surfactant;
at least one amphoteric surfactant; and
at least one dihydroxyacetone.

32. A method of sunless tanning comprising:
applying to skin, a composition comprising:
at least one phospholipid;
at least one nonionic surfactant;
at least one amphoteric surfactant; and
at least one sunless tanning active ingredient.

33. A method of making a sunless tanning composition comprising:
combining:
at least one phospholipid;
at least one nonionic surfactant;
at least one amphoteric surfactant; and
at least one sunless tanning active ingredient.

34. A composition, comprising:
at least one phospholipid;
at least one nonionic surfactant;
at least one amphoteric surfactant; and
at least one dihydroxyacetone;
wherein the composition is an aqueous-based solution.
